SEQUENCE LISTING (25 sequences)

SEQ ID NO: 1 - DNA, 1314 bp, Arthrobacter crystallopoietes
  CDS (1)..(1314)
atggatgcgaaactccttgttggcggcactattgtttcctcgaccggcaaaatccgsgccgacgtgctgattgaaaacggcaaagtcgccgctgtcggcatgctggacgccgcgacgccggacacagttgagcgggttgactgcgacggcsaatacgtcatgcccggcggtatcgacgttcacacccacatcgactcccccctcatggggaccaccaccgccgatgattttgtcagcggaacgattgcagccgctaccggcggaacaacgaccatcgtcgatttcggacagcagctcgccggcaagaacctgctggaatccgcagacgcgcaccacaaaaaggcgcaggggaaatccgtcattgattacggcttccatatgtgcgtgacgaacctctatgacaatttcgattcccatatggcagaactgacacaggacggaatctccagtttcaaggtcttcatggcctaccgcggaagcctgatgatcaacgacggcgaactgttcgacatcctcaagggagtcggctccagcggtgccaaactatgcgtccacgcagagaacggcgacgtcatcgacaggatcgccgccgacctctacgcccaaggaaaaaccgggcccgggacccacgagatcgcacgcccgccggastcggaagtcgaagcagtcagccgggccatcaagatctcccggatggccgaggtgccgctgtatttcgtgcatctttccacccagggggccgtcgaggaagtagctgccgcgcagatgacaggatggccaatcagcgccgaaacgtgcacccactacctgtcgctgagccgggacatctacgaccagccgggattcgagccggccaaagctgtcctcacaccaccgctgcgcacacaggaacaccaggacgcgttgtggagaggcattaacaccggtgcgctcagcgtcgtcagttccgaccactgccccttctgctttgaggaaaagcagcggatgggggcagatgacttccggcagatccccaacggcgggcccggcgtggagcaccgaatgctcgtgatgtatgagaccggtgtcgcggaaggaaaaatgacgatcgagaaattcgtcgaggtgactgccgagaacccggccaagcaattcgatatgtacccgaaaaagggaacaattgcaccgggctccgatgcagacatcatcgtggtcgaccccaacggaacaaccctcatcagtgccgacacccaaaaacaaaacatggactacacgctgttcgaaggcttcaaaatccgttgctccatcgaccaggtgttctcgcgtggcgacctg

SEQ ID NO: 2 - protein, 438 aa, Arthrobacter crystallopoietes
  misc_feature (51)..(51): The ′Xaa′ at location 51 stands for Glu, or Gln.
MDAKLLVGGTIVSSTGKIRADVLIENGKVAAVGMLDAATPDTVERVDCDGXYVMPGGIDVHTHIDSPLMGTTTADDFVSGTIAAATGGTTTIVDFGQQLAGKNLLESADAHHKKAQGKSVIDYGFHMCVTNLYDNFDSHMAELTQDGISSFKVFMAYRGSLMINDGELFDILKGVGSSGAKLCVHAENGDVIDRIAADLYAQGKTGPGTHEIARPPXSEVEAVSRAIKISRMAEVPLYFVHLSTQGAVEEVAAAQMTGWPISAETCTHYLSLSRDIYDQPGFEPAKAVLTPPLRTQEHQDALWRGINTGALSVVSSDHCPFCFEEKQRMGADDFRQIPNGGPGVEHRMLVMYETGVAEGKMTIEKFVEVTAENPAKQFDMYPKKGTIAPGSDADIIVVDPNGTTLISADTQKQNMDYTLFEGFKIRCSIDQVFSRGDL

SEQ ID NO: 3 - DNA, 26 bp, Artificial Sequence
  Synthetic DNA
gatgttcacgcaccttctttcacttc

SEQ ID NO: 4 - DNA, 25 bp, Artificial Sequence
  Synthetic DNA
ggtgttgtagcccaggacgacgagc

SEQ ID NO: 5 - DNA, 26 bp, Artificial Sequence
  Synthetic DNA
gagggcgatgaagtcgtcgttgtgaa

SEQ ID NO: 6 - DNA, 25 bp, Artificial Sequence
  Synthetic DNA
gttctggtatgcccctgcctgaagt

SEQ ID NO: 7 - DNA, 26 bp, Artificial Sequence
  Synthetic DNA
aacatatggcgaaaaacttgatgctc

SEQ ID NO: 8 - DNA, 26 bp, Artificial Sequence
  Synthetic DNA
aaggatccgtcattcacgttgaacgg

SEQ ID NO: 9 - DNA, 29 bp, Artificial Sequence
  Synthetic DNA
aaggatccttagtcattcacgttgaacgg

SEQ ID NO: 10 - DNA, 17 bp, Artificial Sequence
  Synthetic DNA
gtnatgtaygaracvgg

SEQ ID NO: 11 - DNA, 17 bp, Artificial Sequence
  Synthetic DNA
gtrtartccatrttytc

SEQ ID NO: 12 - protein, 13 aa, Artificial Sequence
  Synthetic Peptide
SLVMYETGVAEGK

SEQ ID NO: 13 - protein, 11 aa, Artificial Sequence
  Synthetic Peptide
QNMDYTLFEGK

SEQ ID NO: 14 - DNA, 23 bp, Artificial Sequence
  Synthetic DNA
atcgtggtcgaccccaacggaac

SEQ ID NO: 15 - DNA, 24 bp, Artificial Sequence
  Synthetic DNA
gcatcggagcccggtgcaattgtt

SEQ ID NO: 16 - DNA, 22 bp, Artificial Sequence
  Synthetic DNA
atgcggtcgcaaccacaaccca

SEQ ID NO: 17 - DNA, 22 bp, Artificial Sequence
  Synthetic DNA
agcgccagggccggaagaagca

SEQ ID NO: 18 - DNA, 19 bp, Artificial Sequence
  Synthetic DNA
ggcccattttcctgtcagt

SEQ ID NO: 19 - DNA, 18 bp, Artificial Sequence
  Synthetic DNA
aggctgaaaatcttctct

SEQ ID NO: 20 - DNA, 28 bp, Artificial Sequence
  Synthetic DNA
aacatatggaaacaattgacggcatttc

SEQ ID NO: 21 - DNA, 26 bp, Artificial Sequence
  Synthetic DNA
aaggatccgggccgtgactcgtcgac

SEQ ID NO: 22 - DNA, 45 bp, Artificial Sequence
  Synthetic DNA
aaaaggatccgaaggagatatacaatggangcgaaactccttgtt

SEQ ID NO: 23 - DNA, 33 bp, Artificial Sequence
  Synthetic DNA
aaaaaagcttctaccgcttgatgaattcgccgc

SEQ ID NO: 24 - DNA, 61 bp, Artificial Sequence
  Synthetic DNA
aaaagcttttatttttcgaactgcgggtcgctccaagcgctccgcttgatgaattcgcccg

SEQ ID NO: 25 - DNA, 26 bp, Artificial Sequence
  Synthetic DNA
aacatatgctcgcggtcgctcaagtc

(19) United States
(12) Patent Application Publication
(10) Pub. No.: US 2003/0143677 A1
(43) Pub. Date: Jul. 31, 2003

(54) Activated rec-D-hydantoinases

(75) Inventors: Oliver May, Frankfurt (DE); Andreas Bommarius, Atlanta, GA (US); Karlheinz Drauz, Freigericht (DE); Martin Siemann-Herzberg, Wildberg (DE); Christoph Syldatk, Stuttgart (DE); Markus Werner, Weinsberg (DE); Josef Altenbuchner, Nufringen (DE)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: DEGUSA AG, Duesseldorf (DE)

(21) Appl. No.: 10/176,584

(22) Filed: Jun. 24, 2002

(30) Foreign Application Priority Data
Jun. 22, 2001 (DE) 101 30 169.3

(51) Int. Cl. C12P 21/02; C12N 1/21; C07H 21/04; C12N 9/86; C12N 15/74

(52) U.S. Cl. 435/69.1; 435/231; 435/252.3; 435/320.1; 536/23.2

(57) ABSTRACT

Rec-hydantoinases which may be obtained in more active form by a the process described herein. The invention also relates, inter alia, to a rechydantoinase from the organism Arthrobacter crystallopoietes DSM20117, to nucleic acids which code for such a protein and to vectors containing said nucleic acids and to uses thereof.

### BACKGROUND OF THE INVENTION

1. Field of the Invention

The present invention relates to rec-hydantoinases which may be obtained in more active form by the process described herein. The invention also relates, inter alia, to a rec-hydantoinase from the organism Arthrobacter crystallopoietes DSM20117, to nucleic acids which code for such a protein, and to vectors containing these nucleic acids.

2. Background of the Invention

The use of enzymatic processes in the industrial-scale synthesis of organic compounds is well established, as such processes are frequently superior to conventional chemical processes with regard to selectivity and product yields. Enantiomer-enriched amino acids, in particular, are preferred targets for the use of processes which operate enzymatically, but these processes are also of vital significance in the natural world, for example in the biosynthesis of amino acids and proteins. Enantiomer enriched amino acids are also important products in relation to the synthesis of bioactive compounds or in parenteral nutrition.

Hydantoinases are enzymes which are capable of converting 5′-substituted hydantoins, optionally stereoselectively, into L- or D-N-carbamoylamino acid (FIG. 1). Racemic, 5′-substituted hydantoins may preferably be obtained very straightforwardly by chemical synthesis (Kleinpeter, Structural Chemistry 1997, 8, 161-173; Ogawa et al., Tibtech 1999, 17, 1039-43; Beller et al., Angew. Chem. 1999, 111, 1562-65). The targeted processes for the production of enantiomer-enriched amino acids are accordingly preferably performed on an industrial scale (Drauz K, Kottenhahn M, Makryaleas K, Klenk H, Bemd M, Angew Chem, (1991). Chemoenzymatic synthesis of D- -ureidoamino acids, 103, 704-706; FIG. 2).

Screening for hydantoin-utilizing microorganisms has previously resulted in the isolation of both Gram-positive and Gram-negative prokaryotes from five different phylogenetic groups: Alcaligenes, Arthrobacter, Bacillus, Blastobacter, Flavobacterium, Nocardia, Pseudomonas, Comamonas, Thermus and Agrobacterium. The complete arrangement of the genes which code for the enzymes involved in degradation has hitherto been described only for three organisms. The coding structural genes of these enzymes are arranged adjacent to each other in the form of a “hyu gene cluster” (hyu denotes “hydantoin utilizing”; after Watabe et al., J. Bacteriol. 1992, 174, 3461-66; ibid, 962-969) on the genomic DNA (Arthrobacter and Agrobacterium) or on a plasmid (Pseudomonas). Of the three hyu gene clusters, Agrobacterium and Pseudomonas contain the genes for D-selective cleavage and Arthrobacter contains the genes for L-selective cleavage (Hils, thesis, University of Stuttgart, 1998; Watabe et al., op. cit.; Wiese, thesis, University of Stuttgart, 2000, Verlag Ulrich Grauer).

It was also known that the organism Arthrobacter crystallopoietes DSM 20117 has a D-hydantoinase (Syldatk et al. in Jahrbuch Biotechnologie, volume 2, 1988/1989, ed.: P. Präve). It has already been possible to elucidate the N-terminal sequence of the enzyme (A. Marin, thesis, University of Stuttgart, 1997).

However, it has not hitherto been possible to achieve production of the described hydantoinase in recombinant and active form (M. Werner, thesis, University of Stuttgart 2001).

### SUMMARY OF THE INVENTION

The object of the present invention is to provide a process for the recombinant production of active hydantoinases and to provide the active recombinant hydantoinases obtained using this process.

The present invention is based, inter alia, on the discovery that fermenting a microorganism which produces a rec-hydantoinase in the presence of divalent metal ions provides an activated rec-hydantoinase.

Accordingly, the present invention provides a process for producing an activated rec-hydantoinase, comprising fermenting a microorganism which produces a rec-hydantoinases in the presence of a concentration of at least one divalent metal ion sufficient to activate the rec-hydantoinase.

The present invention also provides an activated rec-hydantoinase obtainable by the process described above.

The present invention also provides an activated rec-hydantoinase obtained by a process comprising fermenting a microorganism which produces a rec-hydantoinase in the presence of a concentration of at least one divalent metal ion sufficient to activate the rec-hydantoinase.

The present invention also provides an isolated nucleic acid which codes for a D-hydantoinase from Arthrobacter crystallopoietes DSM 20117.

The present invention also provides a plasmid, vector, or microorganism comprising the nucleic acid described above.

The present invention also provides nucleic acids which hybridizes with the single-stranded nucleic acid or complementary single-stranded nucleic acid described above under stringent conditions.

In addition, the present invention provides primers suitable for producing the nucleic acid described above by means of PCR.

The present invention also provides process for the producing an improved rec-hydantoinase, comprising:

(a) mutagenizing a nucleic acid which codes for a rec-hydantoinase,

(b) cloning the mutangenized nucleic acids from (a) into a vector,

(c) transferring the vector from (b) into an expression system,

(d) expressing the nucleic acid in the expression system,

(e) detecting protein which have improved activity and/or selectivity, and

(f) isolating the protein detected in (e).

The present invention also provides a rec-hydantoinases obtainable by the process described above.

The present invention also provides a process for the producing a nucleic acid which encodes an improved rec-hydantoinase, comprising:

(a) mutagenizing a nucleic acid which codes for a rec-hydantoinase,

(b) cloning the mutangenized nucleic acids from (a) into a vector,

(c) transferring the vector from (b) into an expression system,

(d) expressing the nucleic acid in the expression system,

(e) detecting protein which have improved activity and/or selectivity, and

(f) isolating the nucleic acid which encodes the protein detected in (e).

In addition, the present invention provides a nucleic acid obtainable by the process described above.

The present invention also relates to a method of producing an N-carbamoylamino acid, comprising

contacting a hydantoin with the the rec-hydantoinase described above.

The present invention also relates to a method of making an amino acid, comprising:

producing an N-carbamoylamino acid as described above, and

contacting the N-carbamoylamino acid with a carbamoylase.

A more complete appreciation of the invention and many of the attendant advantages thereof will be readily obtained as the same becomes better understood by reference to the following Figures in conjunction with the detailed description below.

### BRIEF DESCRIPTION OF THE FIGURES

FIG. 1: scheme showing the enzymatic conversion of 5′-substituted hydantoins into L- or D-N-carbamoylamino acids.

FIG. 2: scheme showing the production of enantiomer-enriched amino acids.

FIG. 3: restriction map for plasmid pCR-BluntII.

FIG. 4: restriction map for plasmid pJOE 4036.

FIG. 5: restriction map for plasmid pJOE 3078.4

FIG. 6: restriction map for plasmid pMW10.

FIG. 7: D-hyd specific activity results obtained in the Examples described below.

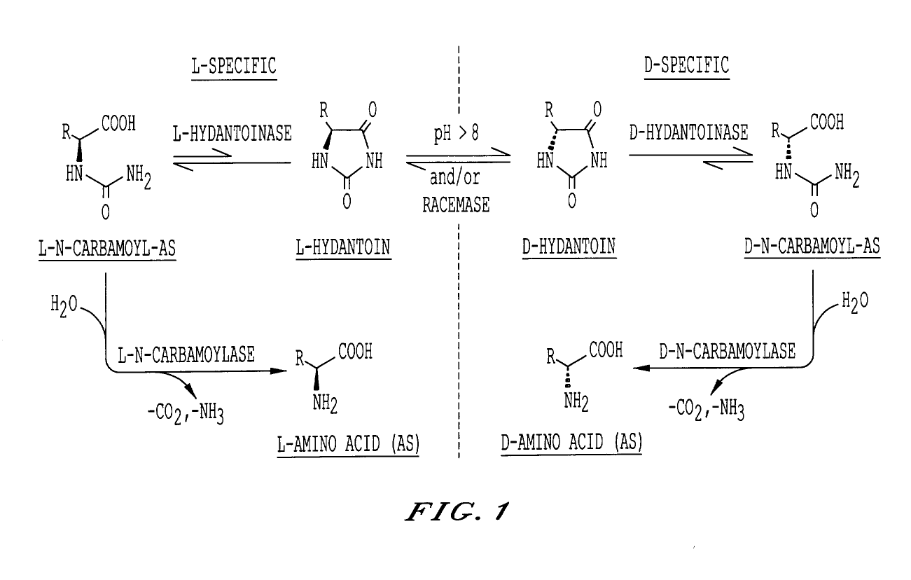

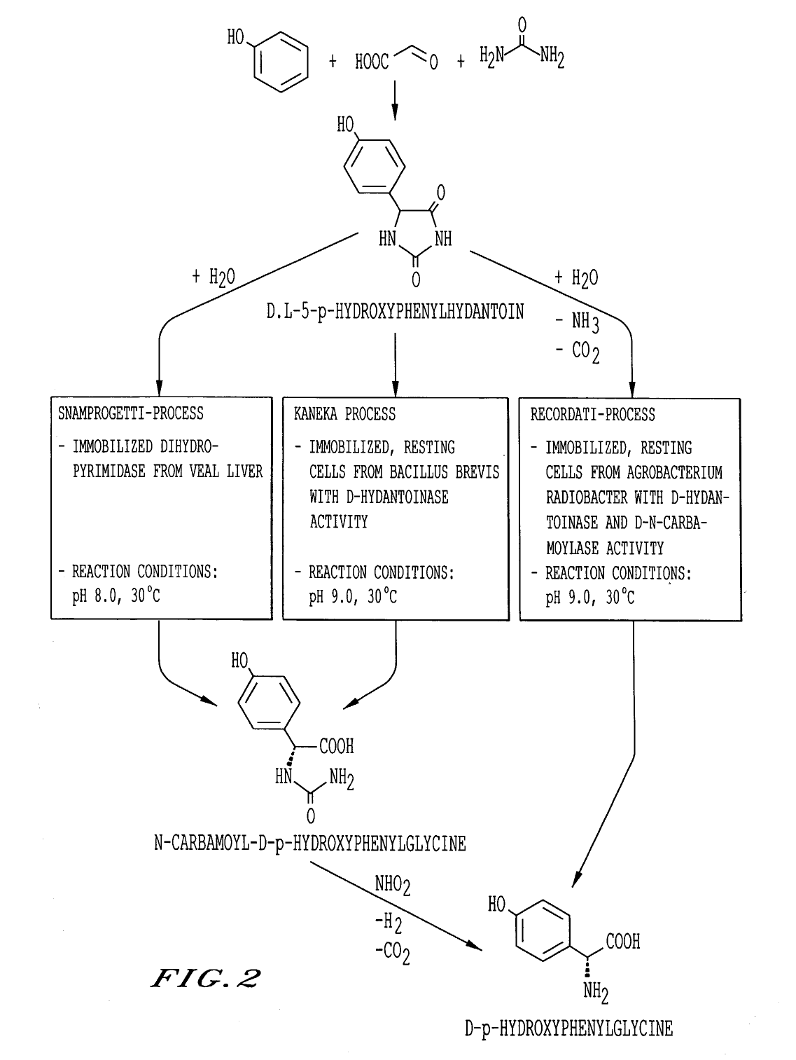

### DETAILED DESCRIPTION OF THE INVENTION

In a process for the production of activated rec-hydantoinases by fermentation of the microorganisms which form the rec-hydantoinases in the presence of divalent metal ions, the fact that the fermentation broth comprises a concentration of metal ions (for example Co, Mn, Zn) which brings about activation results in a surprisingly simple but consequently no less advantageous manner in rec-hydantoinases which exhibit increased specific activity (activation) in comparison with rec-hydantoinases which are formed by rec-hydantoinase expressing microorganisms fermented under otherwise conventional conditions. It must be considered surprising that in particular increasing the concentration of zinc ions, which is actually disadvantageous for the growth of the microorganisms during fermentation (longer growth period is required), should bring about a considerable increase in activity.

The fermentation broth thus preferably comprises a concentration of zinc ions which brings about the increase in activity. The optimum concentration at which the metal ions, in particular zinc ions, are added to the fermentation broth may be determined by one skilled in the art by means of routine experimentation that concentration should, on the one hand, be selected such that it is high enough to achieve an activation/increase in activity according to the invention, but, on the other, should not be so high that growth of the microorganisms is excessively inhibited without creating a further increase in activity. The concentration of metal ions, for example zinc ions, during fermentation should preferably be raised to 30 μmol/l, particularly preferably to 50 μmol/l and most particularly preferably to 80 μmol/l. The rec-hydantoinase under consideration particularly preferably comprises the hydantoinase from Arthrobacter crystallopoietes DSM20117.

In a further development, the invention relates to rec-hydantoinases obtainable by the process according to the invention. Without being limited to any particular theory, it is to be assumed that, although the activated and unactivated enzymes match in terms of their primary structure, the increase in zinc ion concentration during fermentation probably has an influence upon the formation of the secondary or even tertiary structure of the enzymes such that an improvement in the specific activity of the proteins is achieved.

In a preferred embodiment, the invention relates to nucleic acids coding for a D-hydantoinase from Arthrobacter crystallopoietes DSM 20117.

By providing the nucleic acids which code for a D-hydantoinase from Arthrobacter crystallopoietes DSM 20117, substances are advantageously obtained which make it possible to provide a sufficient quantity of the enzymes required for an industrial enzymatic process for the production of D-amino acids. By using recombinant methods, it is possible with the nucleic acids to obtain the enzymes at high yield from rapidly growing host organisms.

Moreover, the gene sequences according to the invention are be used to produce improved hydantoinase mutants.

In another embodiment, the invention relates to plasmids or vectors comprising one or more of the nucleic acids according to the invention.

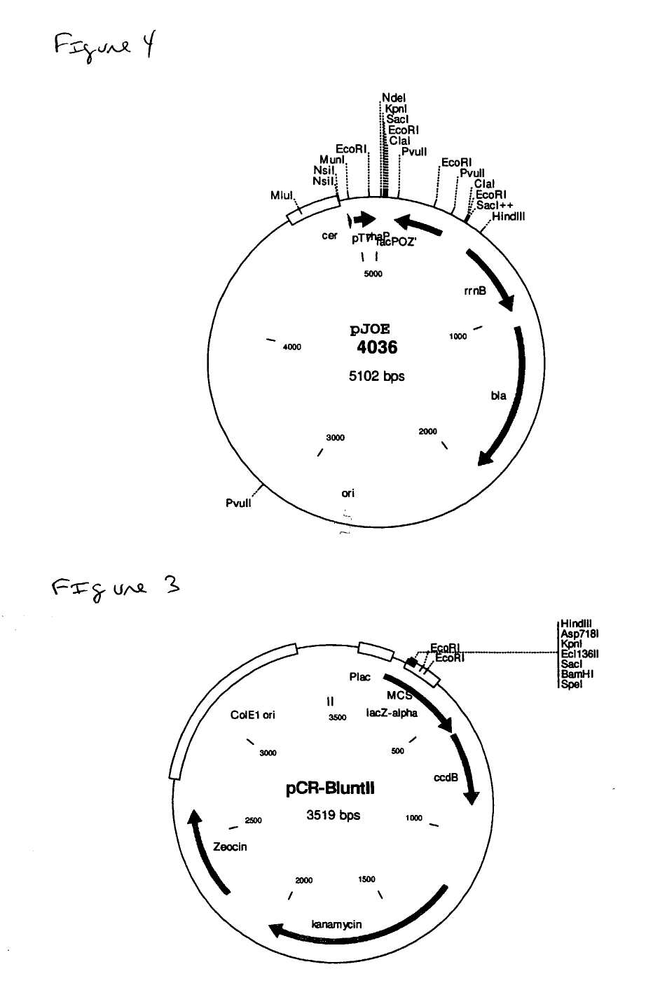

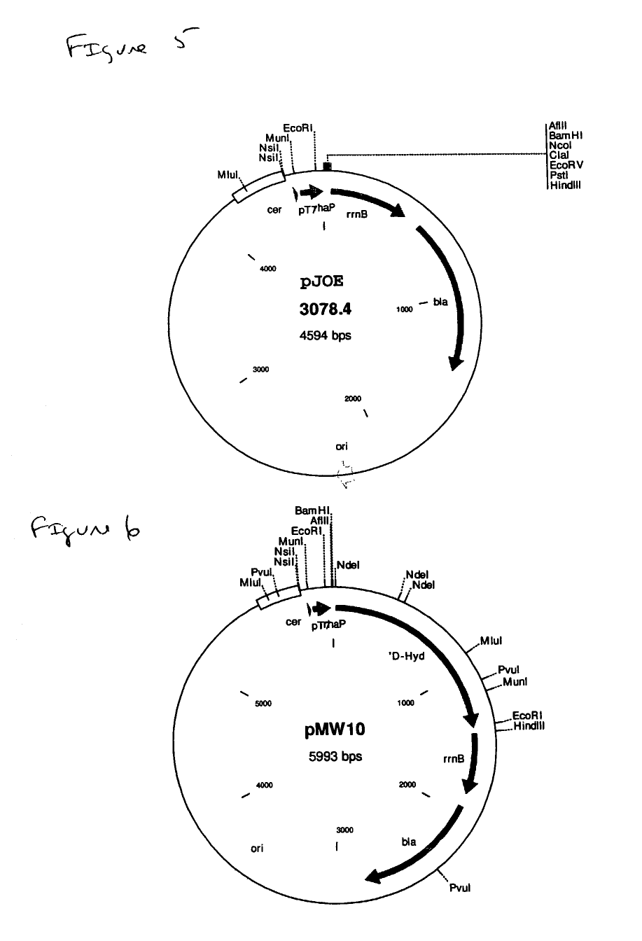

Plasmids or vectors which may be considered are in principle any types available to one skilled in the art for this purpose. Such plasmids and vectors may be found in Studier et al., Methods Enzymol. 1990, 185, 61-69 or in brochures from the companies Novagen, Promega, New England Biolabs, Clontech or Gibco BRL. Further preferred plasmids and vectors may be found in: DNA cloning: a practical approach. Volume I-III, edited by D. M. Glover, IRL Press Ltd., Oxford, Wash. D.C., 1985, 1987; Denhardt, D. T. and Colasanti, J.: A survey of vectors for regulating expression of cloned DNA in E. coli. in: Rodriguez, R. L. and Denhardt, D. T (eds), Vectors, Butterworth, Stoneham, Mass., 1987, pp179-204; Gene expression technology. in: Goeddel, D. V. (eds), Methods in Enzymology, volume 185, Academic Press, Inc., San Diego, 1990; Sambrook, J., Fritsch, E. F. and Maniatis, T. 1989. Molecular cloning: a laboratory manual, 2nd ed., Cold Spring Harbor Laboratory Press, Cold Spring Harbor, N.Y. Plasmids with which the gene construct comprising the nucleic acid according to the invention may very preferably be cloned into the host organism are: pKK-177-3H (Roche Biochemicals), pBTac (Roche Biochemicals), pKK-233 (Stratagene) or pET (Novagen). With the exception of the TOPO series, which has integral kanamycin resistance, all the other plasmids should contain a β-lactamase for ampicillin resistance. The following are very particularly preferred plasmids:

| Designation | Characteristics | Primer involved |
|---|---|---|
| pJW2 | pCRTOPOBluntII (FIG. 3) with | IPCR1+/− |
| | amplicon from IPCR 1 | |
| pRW | pCRTOPOBluntII with amplicon from | IPCR1+/5− |
| | IPCR 2 | |
| pMW1 | PJOE4036 (FIG. 4) + DC (ttg start) | K_DCn2/c2 |
| | with His-tag | |
| pMW2 | PJOE4036 + DC (ttg start) without | K_DCn2/c3 |
| | His-tag | |
| pMW3 | PJOE4036 + DC (atg start) with His-tag | K_DCn1/c2 |
| pMW10 | PJOE3078 (FIG. 5) + DHP with Strep- | K_DHPn2/c5 |
| | tag | |
| pMW11 | PJOE3078 + DHP without Strep-tag | K_DHPn2/c2 |
| pJW1 | pCRTOPOBluntII with hydantoinase | 51.61a/73.31b |
| | fragment from PCR with degenerate | |
| | primers | |
| pCF1 | pCRTOPOBluntII with amplicon from | IPCR1+/− |
| | IPCR 3 | |
| pCF2 | pCRTOPOBluntII with amplicon from | IPCR1+/− |
| | IPCR 4 | |

All of the publications cited above are incorporated herein by reference.

The invention also relates to microorganisms comprising the nucleic acids according to the invention.

The purpose of the microorganism into which the nucleic acids are cloned is to multiply and to obtain a sufficient quantity of the recombinant enzyme. The methods used for this, purpose are well-known to one skilled in the art (Sambrook et al. 1989, Molecular cloning: A Laboratory Manual, 2nd Edition, Cold Spring Harbor Laboratory Press, Balbas P & Bolivar F. 1990, Design and construction of expression plasmid vectors in E. coli, Methods Enzymology 185, 14-37, both incorporated herein by reference). Microorganisms which may be used are in principle any organisms known by one skilled in the art for this purpose. Strains of E. coli should preferably be used for this purpose. The following are very particularly preferred: E. coli NM 522, JM109, JM105, RR1, DH5 , TOP 10- or HB101. Plasmids with which the gene construct comprising the nucleic acid according to the invention is preferably cloned into the host organism are described above.

The invention also relates to nucleic acids which, under stringent conditions, hybridize with the single-stranded nucleic acids according to the invention or the single stranded nucleic acids complementary thereto. The phrase “under stringent conditions” is used herein as described in Sambrook et al. (Molecular Cloning, A Laboratory Manual, Cold Spring Harbor Laboratory Press (1989), 1.101-1.104), incorporated herein by reference. Stringent hybridization according to the present invention is preferably obtained when a positive hybridization signal is still observed after washing for 1 hour with 1×SSC and 0.1% SDS (sodium dodecyl sulfate) at 50° C., preferably at 55° C., more preferably at 62° C. and most preferably at 68° C. and more preferably for 1 hour with 0.2×SSC and 0.1% SDS at 50° C., more preferably at 55° C., still more preferably at 62° C. and most preferably at 68° C.

Another aspect of the invention below relates to primers for the production of the gene sequences according to the invention by means of all kinds of PCR. This includes both sense and antisense primers which code for the corresponding amino acid sequences.

Suitable primers may in principle be obtained using methods known to one skilled in the art. The primers according to the invention are identified by comparison with known DNA sequences or by translation of the amino acid sequences under consideration into the codon of the organism in question (for example for Streptomyces: Wright et al., Gene 1992, 113, 55-65). Common features in the amino acid sequence of proteins from “superfamilies” may also be exploited for this purpose (Firestine et al., Chemistry & Biology 1996, 3, 779-783, incorporated herein by reference). Further information in this connection may be found in Oligonucleotide Synthesis: a Practical Approach, edited by M. J. Gait, IRL Press Ltd, Oxford Wash. D.C., 1984; PCR Protocols: A guide to methods and applications, edited by M. A. Innis, D. H. Gelfound, J. J. Sninsky and T. J. White. Academic Press, Inc., San Diego, 1990, all incorporated herein by reference. The following primers are extremely preferred:

Primers for IPCR:

Des-
ig-
na-

| | | |
|---|---|---|
| tion | Sequence | Seq. |

| | | |
|---|---|---|
| IPC- | 5′-AT GTT CAC GCA CCT TCT TTC ACT TC-3′ | 3 |
| R1+ | | |
| IPC- | 5′-GT GTT GTA GCC GAG GAG GAG GAG C-3′ | 4 |
| R1− | | |
| IPC- | 5′-GAG GGC GAT GAA GTC GTC GTT GTG AA-3′ | 5 |
| R5+ | | |
| IPC- | 5′-TT CTG GTA TGC CCC TGC CTG AAG T-3′ | 6 |
| R5− | | |
| IPC- | 5′-TC GTG GTC GAG CCC AAC GGA AC-3′ | 14 |
| R7+ | | |
| IPC- | 5′-GCA TCG GAG CCC GGT GCA ATT GTT-3′ | 15 |
| R7− | | |
| IPC- | 5′-TG CGG TCG CAA CCA CAA CCC A-3′ | 16 |
| R11+ | | |
| IPC- | 5′-GC GCC AGG GCC GGA AGA AGC A-3′ | 17 |
| R11− | | |

Primers for Cloning Structural Genes:

| | | |
|---|---|---|
| Designation | Sequence | Seq. |

| | | |
|---|---|---|
| K_DCn2 | 5′-AAC ATA TGG CGA AAA ACT TGA TGC TC-3′ | 7 |
| K_DCc2 | 5′-AAG GAT CCG TCA TTC ACG TTG AAC GG-3′ | 8 |
| K_DCc3 | 5′-AAG GAT CCT TAG TCA TTC ACG TTG AAC GG-3′ | 9 |
| K_LCn1 | 5′-AAC ATA TGG AAA CAA TTG ACG GCA TTT C-3′ | 20 |
| K_LCc1 | 5′-AAG GAT CCG GGC CGT GAC TCG TCG AC-3′ | 21 |
| K_DHP n2 | 5′-AAAA GGATCC GA AGGAGA TATACA ATG | 22 |
| | GAZGCGAAACTCCTTGTT-3′ | |
| K_pHPc2 | 5′-AAAA AAGCTT CTA CCGCTTGATGAATTCGCCGC-3′ | 23 |
| K_DHPc5 | 5′-AA AAGCTT TTA TTT TTC GAA CTG CGG GTG G CT | 24 |
| | CCA AGC GCT CCGCTTGATGAATTCGCCCG-3′ | |
| K_DCn1 | 5′-AAC ATA TGC TCG CGG TCG CTC AAG TC-3′ | 22 |

Primers for Sequencing DNA in Rhamnose Expression Vectors:

| Designation | Sequence | Seq. |
|---|---|---|
| S1995 (n-term) | 5′-GGC CCA TTT TCC TGT CAG T-3′ | 18 |
| S998 (c-term) | 5′-AGG CTG AAA ATC TTC TCT-3′ | 19 |

In another embodiment, the present invention relates to a process for the production of improved rec-hydantoinases and to rec-hydantoinases obtained in this manner or to nucleic acids which code for the latter, wherein, starting from the nucleic acids according to the invention which code for a rec-hydantoinase, a) the nucleic acids are subjected to mutagenesis, b) the nucleic acids obtainable from a) are cloned into a suitable vector and the latter is transferred into a suitable expression system and c) the proteins formed having improved activity and/or selectivity are detected and isolated.

The procedure for improving the enzymes according to the invention by mutagenesis methods is known to one skilled in the art. Mutagenesis methods which may be considered are any methods available to one skilled in the art for this purpose. In particular, these methods are saturation mutagenesis, random mutagenesis, shuffling methods and site-directed mutagenesis (see below for literature). The resultant novel nucleic acid sequences are cloned into a host organism using the methods described above (see above for literature) and the expressed enzymes are detected with suitable screening methods (Roberts J., Stella V. J. and Decedue C. J. (1985) A colorimetric assay of pancreatic lipase: rapid detection of lipase and colipase separated by gel filtration. Lipids 20(1): 42-45; Pratt R. F., Faraci W. S. and Govardhan C. P. (1985) A direct spectrophotometric assay for D-alanine carboxypeptidases and for the esterase activity of beta-lactamases. Anal. Biochem. 144(1): 204-206; Bruckner, H., R. Wittner, and H. Godel (1991), all incorporated herein by reference. Fully automated high-performance liquid chromatographic separation of DL-amino acids derivatized with o-phthaldialdehyde together with N-isopropyl-cysteine. Application to food samples).

The present invention also relates to the use of the rec-hydantoinases, optionally improved by mutation, according to the invention for the production of N-carbamoylamino acids or amino acids.

The nucleic acids according to the invention and moreover further improved nucleic acids, which code for the rec-hydantoinases under consideration, are furthermore preferably suitable for the production of whole cell catalysts (DE10037115.9 and literature cited therein, all incorporated herein by reference).

The nucleic acids according to the invention may thus preferably be used for the production of rec-hydantoinases. By means of recombinant methods, which are sufficiently known to the person skilled in the art, organisms are obtained which are capable of providing the enzyme under consideration in a quantity sufficient for an industrial process. The rec-enzymes according to the invention are produced using methods of genetic engineering known to the person skilled in the art (Sambrook J, Fritsch E F, Maniatis T (1989). Molecular Cloning. Cold Spring Harbor Laboratory Press; Vectors: A Survey of Molecular Cloning Vectors and Their Uses. R. L. Rodriguez & D. T. Denhardt, eds.: 205-225). With regard to general procedures (PCR and fusion PCR, inverse PCR, cloning, expression, etc.), reference is made to the following literature and that cited therein: Riley J, Butler R, Finniear R, Jenner D, Powell S, Anand R, Smith J C, Markham A F (1990). A novel, rapid method for the isolation of terminal sequences from yeast artificial chromosome (YAC) clones. Nucl Acids Res. 18, 8186; Triglia T, Peterson M G, Kemp D J (1988), all incorporated herein by reference. A procedure for in vitro amplification of DNA segments that lie outside the boundaries of known sequences. Nucleic Acids Res. 16, 8186; Sambrook J, Fritsch E F, Maniatis T (1989). Molecular Cloning. Cold Spring Harbor Laboratory Press; Vectors: A Survey of Molecular Cloning Vectors and Their Uses. R. L. Rodriguez & D. T. Denhardt, II), all incorporated herein by reference.

As described above, the nucleic acids according to the invention may also be used for the production of novel mutants of the present hydantoinase. Such mutants may be obtained from the DNA according to the invention by known types of mutation. Preferred types of mutation which are to be used are mentioned in the following literature references: (Eigen M. and Gardinger W. (1984) Evolutionary molecular engineering based on RNA replication. Pure & Appl. Chem. 56(8), 967-978; Chen & Arnold (1991) Enzyme engineering for nonaqueous solvents: random mutagenesis to enhance activity of subtilisin E in polar organic media. Bio/Technology 9, 1073-1077; Horwitz, M. And L. Loeb (1986) “Promoters Selected From Random DNA-Sequences” Proceedings Of The National Academy of Sciences Of The United States Of America 83(19): 7405-7409; Dube, D. And L. Loeb (1989) “Mutants Generated By The Insertion Of Random Oligonucleotides Into The Active-Site Of The Beta-Lactamase Gene” Biochemistry 28(14): 5703-5707; Stemmer P C (1994). Rapid evolution of a protein in vitro by DNA shuffling. Nature. 370; 389-391 and Stemmer PC (1994) DNA shuffling by random fragmentation and reassembly: In vitro recombination for molecular evolution. Proc Natl Acad Sci USA. 91; 1074710751). All of the publications cited above are incorporated herein by reference.

At 43% identity of the amino acids, the gene for the hydantoinase according to the invention exhibits the greatest homology with a hypothetical protein from Streptomyces coelicolor (T28685), to which it has, however, not hitherto been possible to assign a function. Levels of identity with the dihydropyrimidinases from Bacillus stearothernophilus (JC2310: Mukohara et al., 1994), Agrobacterium radiobacter NRRLB11291 (Q44184: Grifantini et al., 1996) and Pseudomonas (Stover et al. 2000, La Pointe et al. 1998) are 40%, 42% and 39% respectively.

In addition to homologies with eukaryotic dihydropyrimidinases (from Mus musculus, Homo sapiens and Rattus norvegicus) and collapsin response mediator protein 3 (CRMP-3), there are also homologies with various allantoinases and dihydroorotases.

Identity with the L-hydantoinases from Arthrobacter aurescens DSM 3745 (May et al., Biol. Chem. 1998, 379, 743747, incorporated herein by reference) and DSM 3747 (Wiese, thesis, University of Stuttgart, 2000, incorporated herein by reference) is 29%.

For the purposes of the invention, optically enriched (enantiomer-enriched) compounds are taken to mean the presence of one optical antipode in a mixture with the other in an amount of >50 mol %.

Hydantoins are also intended to mean the compounds derived from 2,4-dioxoimidazolidines, which compounds are substituted in position 5 by a residue which may be derived from the α-residue of an amino acid.

An α-residue of an amino acid is taken to mean the residue located on the α-C atom of an α-amino acid. The residue may be derived from a natural amino acid, as explained in Beyer-Walter, Lehrbuch der organischen Chemie, S. Hirzel Verlag Stuttgart, 22nd edition, 1991, pp. 822 et seq., incorporated herein by reference. However, corresponding α-residues of non-natural α-amino acids, as listed for example in DE19903268.8, incorporated herein by reference, are also included.

The organism Arthrobacter crystallopoietes DSM 20117 has been deposited with Deutsche Sammlung für Mikroorganismen und Zellkulturen under the stated number and is publicly accessible.

In the present invention, the term “activated” or “activation” should be taken to mean that the rec-enzyme according to the invention has, in comparison with the unactivated rec-enzyme at an identical OD₆₀₀, an activity which is increased by a factor of at least 1.5, preferably of 2, particularly preferably of 5 (measurement conditions as in Example VI) in the cell extract (supernatant after 15000 psi, 60 sec, centrifugation at 10000 rpm for 10 min at 4° C.).

The term nucleic acids is taken to subsume all kinds of single-stranded or double-stranded DNA and RNA or mixtures thereof.

In the present invention, improved rec-hydantoinases are in particular taken to mean those which exhibit a modified substrate range, are more active and/or more selective or are more stable under the reaction conditions used.

According to the invention, the claimed protein sequences and nucleic acid sequences also include such sequences which exhibit homology (excluding natural degeneration) with one of these sequences of greater than 80%, preferably of greater than 90%, 91%, 92%, 93% or 94%, more preferably of greater than 95% or 96% and particularly preferably of greater than 97%, 98% or 99%, providing that the mode of action or purpose of such a sequence is retained. The term “homology” (or identity) as used in the present document may be defined by the equation H (%)=[1-V/X]×100, in which H means homology, X is the total number of nucleobases/amino acids of the comparison sequence and V is the number of different nucleobases/amino acids in the sequence under consideration relative to the comparison sequence.

### EXAMPLES

Having generally described this invention, a further understanding can be obtained by reference to certain specific examples which are provided herein for purposes of illustration only and are not intended to be limiting unless otherwise specified.

I. Isolation of Biomass from Arthrobacter crystallopoietes DSM 20117

The aim was initially to prepare a sufficient quantity of a physiologically uniform cell mass of Arthrobacter crystallopoietes DSM 20117 as the starting material for whole cell activity tests, for isolating chromosomal DNA and for enzyme isolation of the D-hydantoinase. To this end, on the basis of the work by Brans (doctoral thesis, T U Braunschweig, 1991), a semi-synthetic medium comprising D,L-lactate as carbon source, yeast extract as a further constituent and hydantoin as inducer was used for culturing in a 50 liter bioreactor.

TABLE 1
Values relate to 1 liter of nutrient solution

| | | | |
|---|---|---|---|
| Sodium lactate | Citric acid | 0.75 | g |
| medium pH 7.2 | | | |
| (V = 1 liter) | | | |
| (Brans, 1991) | Yeast extract | 1.0 | g |
| | FeSO4 * 7 H₂O | 0.01 | g |
| | MgSO4 * 7 H₂O | 0.5 | g |
| | CaSO₄ * 7 H₂O | 0.22 | g |
| | MnSO₄ * 7 H₂O | 0.055 | g |
| | ZnSO₄ * H₂O | 0.005 | g |
| | (NH₄)₂SO₄ | 6.0 | g |
| | D,L-Methionine | 0.05 | g |
| | Hydantoin | 1.0 | g |
| | 50% D,L-lactate | 40 | ml |
| | 1 M KH₂PO₄ | 23 | ml |

A first preculture (V=20 ml) was incubated overnight at 30° C. and 110 rpm. The entire preculture was then used to inoculate the second preculture (V=2 l). After 2 days incubation, 1.5 l of the second preculture were used as an inoculum for the fermentation (V=20 l). Since the inducer hydantoin is consumed during growth, it was continuously apportioned with a metering pump such that the hydantoin concentration in the medium remained a constant 0.2 g/l. Once the cells had been harvested, 205 g of wet biomass (WB) were aliquoted and stored at −20° C.

II. Cleaning-Up of the D-hydantoinase from Arthrobacter crystallopoietes DSM 20117

The protocol for cleaning up the D-hydantoinase from Arthrobacter crystallopoietes DSM 20117 is based, with some modifications, on the protein cleaning-up for D-hydantoinase described by Marin (doctoral thesis, University of Stuttgart, 1997, incorporated herein by reference). The cleaning-up stages were, if possible, performed at 4° C. and the hydantoinase activity of the fractions was initially determined by rapid testing using the Ehrlich photometric detection method. Aliquots of the positive samples were then incubated with the standard substrate D,L-benzylhydantoin and the exact activity determined by HPLC.

The biomass obtained from culturing (see I) Arthrobacter crystallopoietes DSM 20117 was initially subjected as a 30% cell suspension to disruption with glass beads in a stirred ball mill. After recording disruption kinetics, protein concentrations of up to 16.5 g/l could be achieved after 20 minutes of disruption. The cell debris and insoluble constituents were removed by centrifugation and the clarified supernatant was used for the subsequent protamine sulfate precipitation. By this means, the viscosity of the solution could be reduced before carrying out streamline DEAF column chromatography.

The proteins bound in the column were eluted by means of a common salt gradient. The active pooled streamline fractions were combined with an identical volume of 2 M (NH₄)₂SO₄ solution for subsequent further separation by means of hydrophobic interaction chromatography (HIC). The fractions with the highest hydantoin activity were then combined and separated from other proteins by anion exchange chromatography on a MonoQ column.

The hydantoinase clean-up data are summarised in Table 2; SDS-PAGE of the cleaned up D-hydantoinase revealed a molecular weight of 50±5 kDa for this enzyme [10% SDSPAGE of the cleaned up D-hydantoinase after concentration of the MonoQ fractions, ProSieve molecular weight marker and L-hydantoinase from A. aurescens DSM 3745 as 49.7 kDa internal standard (May, thesis, University of Stuttgart, 1998), incorporated herein by reference]

TABLE 2
Clean-up data for D-hydantoinaae

| | Vol. | Prot. | Spec. act. | Purification | Yield |
|---|---|---|---|---|---|
| Purification | [ml] | [g/l] | [U/mg] | factor | [%] |
| Cell disruption | 32 | 16 | 1.5 | — | 100 |
| Protamine | 29 | 17 | 1.4 | 0.9 | 89 |
| sulfate | | | | | |
| precipitation | | | | | |
| Combined | 61 | 3.8 | 1.9 | 1.3 | 57 |
| streamline | | | | | |
| fract. | | | | | |
| Ammonium sulfate | 120 | 1.5 | 3.7 | 2.4 | 85 |
| precipitation | | | | | |
| supernatant | | | | | |
| Combined HIC | 30 | 0.8 | 13.3 | 8.8 | 41 |
| fractions | | | | | |
| Combined MonoQ | 19 | 0.4 | 30.1 | 19.8 | 29 |
| fractions | | | | | |

III. Tryptic Digestion of D-hydantoinase

N-terminal sequencing provides reliable sequencing results only for the first 30 amino acids. The sequence stated in Marin's work did not, however, permit derivation of primers. As a consequence, the protein had to be broken down into several peptides by protease digestion in order to obtain further sequence information. Enzymatic fragmentation was carried out with trypsin, an endopeptidase which cuts specifically after the amino acids lysine and arginine. However, it must be anticipated that activity will be reduced in the case of a following acidic amino acid and even that hydrolysis will not occur in the case of a following proline residue. At an average rate of occurrence of lysine and arginine in proteins of 5.7% and 5.4% respectively, complete digestion must be expected to yield an average peptide length of approx. 9 amino acids. The peptide mixture was then separated by quantitative HPLC.

To digest the hydantoinase from Arthrobacter crystallopoietes DSM 20117 with trypsin, the hydantoinase was cleaned-up to the MonoQ fractions as described, then concentrated with an Amicon filter (cut-off 30 kDa) and separated by means of SDS-PAGE. In order to be certain that the protein was indeed the D-hydantoinase, a portion of the gel was transferred onto a membrane by means of a western blot, cut out and the first eight amino acids determined N-terminally. With the exception of position 2, all the amino acids determined matched the N-terminus determined by Marin (thesis, University of Stuttgart, 1997, incorporated herein by reference), such that it may be assumed that the protein isolated in this case was the same enzyme as had already been described and characterized by Marin.

Thereupon, the hydantoinase band was cut directly out of the polyacrylamide gel of the separated MonoQ fractions and subjected to tryptic digestion in situ in accordance with the manufacturer's instructions (Sigma, Steinheim). The peptides were extracted from the gel with acetonitrile and separated one from the other by preparative HPLC. The fractions were dried in a SpeedVac apparatus and then sequenced N-terminally by Edman degradation. In total, in addition to the N-terminus, it proved possible to sequence nine peptides unambiguously. One of the peptide fractions comprised the consensus motif GXXDXHXH of cyclic amidases, which is involved in binding a zinc atom in the active centre (Abendroth et al., Acta Cryst. 2000, D56, 1166-1169, incorporated herein by reference). In those peptide sequences which do not terminate with a lysine (K) or arginine (R), sequencing came to a premature end due to technical problems or inadequate quality or quantity of the samples.

IV. Cloning of the Hyu Gene Cluster

1. Isolation of Chromosomal DNA from Arthrobacter crystallopoietes DSM 20117

The wet biomass (see I) obtained by culturing Arthrobacter crystallopoietes DSM 20117 in lactate medium was also used for isolating chromosomal DNA. High purity, genomic DNA could be isolated after cell lysis and cleaning-up by means of caesium chloride density gradient centrifugation. Quality was verified by recording an absorption spectrum in order to be able to exclude contamination with phenol. DNA concentration, determined photometrically, was 60 μg DNA/ml.

The cDNA was used for restriction digestion and served as a template for PCR.

2. PCR with Degenerate Primers

By sequencing the peptides obtained from tryptic digestion (see III), it proved possible to obtain further sequence information in addition to the N-terminus of the D-hydantoinase. The peptides were aligned with the known protein sequence of Agrobacterium sp. IP I-671 using ClustalX software (Thompson et al. 1997, The ClustalX windows interface: flexible strategies for multiple sequence alignment aided by quality analysis tools. Nucleic Acids Research. 24, 4876-4882, incorporated herein by reference).

In order to derive degenerate primers from the known peptide sequences, sequence portions from two peptides should be selected which have a low degree of degeneracy in their amino acid composition. Peptides 61.61 and 73.31 were selected for this purpose. Primer 61.61a couples to the plus strand and primer 73.31b to the minus strand of the DNA.

TABLE 3

Construction of the degenerate primers

| | | | |
|---|---|---|---|
| | | Primer | |
| Peptide Seq. | Derived DNA sequence | name | Seq. |

| | | | |
|---|---|---|---|
| SLVMYETGVAEGK | 5′-GT(AGCT) ATG TA (CT) GA(AG) AC(AGC) GG-3′ | 61.61a | 10 |
| (61.61 Seq. 12) | | | |
| QNMDYTLFEGK | 5′-GT(AG) TA(AG) TCC AT(AG) TT(CT) TC-3′ | 73.31b | 11 |
| (73.31 Seq. 13) | | | |

In order to bring about a further reduction in the degree of degeneracy of primer 61.61a, account was taken of the frequency distribution of the codons from Arthrobacter sp. on the basis of the CUTG database (Nakamura et al., Nucl. Acids Res. 1999, 27, 292, incorporated herein by reference). As a consequence, the base triplet “GTA” in position 3 of this oligonucleotide could be disregarded for the purposes of primer construction due to the low probability of 10.4% of this codon for the amino acid valine.

In order to estimate the length of the PCR amplicon, the two primers were aligned with the D-hydantoinase from Agrobacterium sp. IP I-671. According to the alignment, the gap between the two oligos is 69 amino acids, such that PCR using the degenerate primers 61.61a and 73.31b should result in a PCR product approx. 207 by in length.

The PCR was prepared in the temperature profile according to the standard batch at an annealing temperature of 42° C. and optimised with regard to magnesium content to a concentration of 2 mM. The PCR batch was then separated in a 3% agarose gel and the size of the bands determined using Imagemaster image analysis software (molecular weight marker D-15 from Novex). The band having a calculated size of 218 by was eluted from the gel and ligated into the pCR TOPO BluntII vector (FIG. 3). The resultant plasmid was designated pJW1. Subsequent sequencing of the vector revealed homologies with already known dihydropyrimidinases, such that the first DNA portion had accordingly been cloned into the structural gene of the D-hydantoinase.

3. Sequencing of the Hyu Gene Cluster by Inverse PCR

The inverse PCR (IPCR) method was used in order to obtain further sequence information from the flanking DNA regions.

The restriction enzymes BamHI, EcoRI, SacI, PstI, BglII, XindIII, SalI, MunI and MluI were used to digest genomic DNA from Arthrobacter crystallopoietes DSM 20117. The digestion products were separated with a 1% agarose gel and immobilized on a nylon membrane by means of a Southern blot.

A suitable probe was produced by radioactively labelling the MunI linearized plasmid pJW1 by nick translation (Nick Translation Kit from Roche Diagnostics) with ³²P-α-ATP and used with the blot for hybridization (molecular weight marker MWM VII).

On the basis of the size of the hybridization signal obtained from the Southern blot, the genomic PstI digestion product (approx. 2000 bp) was used as a template in the following IPCR. To this end, the digestion product was separated on an agarose gel, eluted from the gel in the range between 1500 and 2800 by (molecular weight marker MWM VII), then religated and linearized with MunI. The primers IPCR1+(Seq. 3) and IPCR1 (Seq. 4) could be derived for the IPCR from the known sequence of the hydantoinase gene. The annealing temperature of 60° C. was derived from the melting temperatures of the oligos.

One single band could be generated as the amplicon, which was subsequently eluted and cloned into the TOPO system (FIG. 3). The resultant plasmid was denoted pJW2. The hyu gene cluster reconstructed on the basis of sequencing pJW2 contains the open reading frame of the D-hydantoinase hyuH and part of the open reading frame of the D-carbamoylase hyuCD.

V. Expression of the D-hydantoinase

The D-hydantoinase structural genes were cloned into plasmid derivatives of the rhamnose expression vector pJOE4036 (FIG. 4). The two carbamoylases were amplified by corresponding primers from the genomic DNA of Arthrobacter crystallopoietes. The primers were here equipped at the N-terminus with an additional sequence for an NdeI restriction site and/or a BanM restriction site at the C-terminus. In the case of the enzymes with the His-tag, the stop codon was omitted on the C-terminal primer.

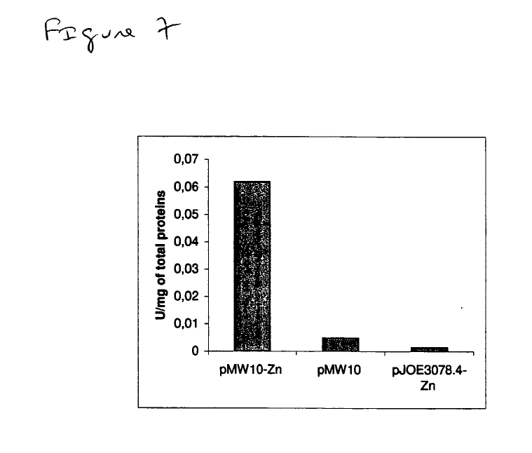

Because the hydantoinase gene contained two internal NdeI restriction sites, the strategy of cloning into pJOE4036 used for the carbamoylases could not be used. Instead, a primer was used which, in addition to the N-terminal DNA sequence, coded at the 51 end for a Shine-Dalgarno sequence and a BamHI restriction site. The C-terminal primer comprised a HindIII restriction site with a stop codon or with a sequence for a Strep-tag. The DNA amplified in this manner from the genomic DNA was then cloned into pJOE3078. The resultant construct was named pMW10 (FIG. 6).

An E. coli containing plasmid pMW10 (FIG. 6) was cultured as follows in LB medium containing 100 μg/ml of ampicillin:

a single colony was transferred in 10 ml of LB medium into a 100 ml shaking flask and incubated overnight at 37° C.

100 ml of the LB medium containing the stated quantity of ampicillin were combined in a 500 ml shaking flask with 2 ml of the culture which had been incubated overnight (batch 1).

An identical quantity of overnight culture, LB medium and ampicillin was introduced into a second shaking flask (500 ml) and additionally combined with 1 ml of a 100 mM ZnSO₄*7H₂O solution (Znz+ conc. in the culture 1 mM) (batch 2).

Batch 1 was incubated for 2 h at 37° C. until the OD₆₀₀ was ˜0.4. Batch 2 took 3 hours to reach the same OD value.

Expression was induced by combining both batches with L-rhamnose until a concentration of 0.1 g/L was obtained. The batches were then cultured for a further 6 h at 30° C.

The cells were then centrifuged for 10 min at 4° C. and 7000 rpm. The pellets obtained were stored at −10° C.

VI. Measurement of Activity

1 g of the stored cells from V. were resuspended in 10 mL portions of 0.1 M phosphate buffer (pH 7.5). The cells were then disrupted at 15000 psi within 60 sec and the resultant suspensions were centrifuged at 10000 rpm for 10 min at 4° C. 25 μl portions of the supernatant were introduced into a preheated sample of 100 μL of 0.1 M phosphate buffer (pH 7.5) containing 8 mM of D-benzylhydantoin. Once the samples had been incubated for 5 min at 50° C., the reaction was terminated. by adding 100 μl of 10% H₃PO₄. After centrifuging again at 10000 rpm for 10 min, 100 pl portions of the supernatant were diluted ten-fold with the mobile phase of the HPLC mobile solvent (0.3% (v/v) phosphoric acid (80%), methanol (20% v/v)). The concentrations of the N-carbamoylamino acids obtained were then determined by means of HPLC.

HPLC method:

Thermoseparation products, Darmstadt, Germany

Gromsil ODS 1 PE column (5 μm, 250×4.6 mm, Grom, Herremberg, Germany)

UV adsorption at 210 nm

Flow rate 1.0 mL/min

Specific activity is defined in units per mg of total protein determined by the Bradford method. One unit of D-hydantoinase catalyses the formation of 1 μmol of carbamoylamino acid starting from D-benzylhydantoin per min at pH 7.5 and 50° C. FIG. 7 shows the D-hyd specific activity results in the cell extract for both batches compared with the microorganism not containing the hyd gene.

It can be seen that the sample fermented in the presence of increased zinc concentrations contains a hydantoinase which is more active by a factor of >12.

Obviously, numerous modifications and variations of the present invention are possible in light of the above teachings. It is therefore to be understood that within the scope of the appended claims, the invention may be practiced otherwise than as specifically described herein.

This application is based on German Patent Application Serial No. 101 30 169.3, filed on Jun. 22, 2001, and incorporated herein by reference.

1. A process for producing an activated rec-hydantoinase, comprising fermenting a microorganism which produces a rec-hydantoinase in the presence of a concentration of at least one divalent metal ion sufficient to activate the rec-hydantoinase.

2. The process of claim 1, wherein the divalent metal ion is zinc ion.

3. The process of claim 1, wherein the divalent metal ion is manganese ion.

4. The process of claim 1, wherein the divalent metal ion is cobalt ion.

5. The process of claim 1, wherein the concentration of the divalent metal ion is at least 30 μmol/l.

6. The process of claim 1, wherein the concentration of the divalent metal ion is at least 50 μmol/l.

7. The process of claim 1, wherein the concentration of the divalent metal ion is at least 80 μmol/l.

8. The process of claim 1, wherein the rec-hydantoinase is from Arthrobacter crystallopoietes DSM 20117.

9. An activated rec-hydantoinase obtainable by the process of claim 1.

10. An activated rec-hydantoinase obtained by a process comprising fermenting a microorganism which produces a rec-hydantoinase in the presence of a concentration of at least one divalent metal ion sufficient to activate the rec-hydantoinase.

11. An isolated nucleic acid which codes for a D-hydantoinase from Arthrobacter crystallopoietes DSM 20117.

12. A plasmid, vector, or microorganism comprising the nucleic acid of claim 5.

13. A nucleic acid which hybridizes with the single-stranded nucleic acid or complementary single-stranded nucleic acid of claim 5 under stringent conditions.

14. A primer suitable for producing the nucleic acid of claim 5 by means of PCR.

15. A process for the producing an improved rec-hydantoinase, comprising:
(a) mutagenizing a nucleic acid which codes for a rec-hydantoinase,
(b) cloning the mutangenized nucleic acids from (a) into a vector,
(c) transferring the vector from (b) into an expression system,
(d) expressing the nucleic acid in the expression system,
(e) detecting protein which have improved activity and/or selectivity, and
(f) isolating the protein detected in (e).

16. A rec-hydantoinase obtainable by the process of claim 15.

17. A process for the producing a nucleic acid which encodes an improved rec-hydantoinase, comprising:
(a) mutagenizing a nucleic acid which codes for a rec-hydantoinase,
(b) cloning the mutangenized nucleic acids from (a) into a vector,
(c) transferring the vector from (b) into an expression system,
(d) expressing the nucleic acid in the expression system,
(e) detecting protein which have improved activity and/or selectivity, and
(f) isolating the nucleic acid which encodes the protein detected in (e).

18. A nucleic acid obtainable by the process of claim 17.

19. A method of producing an N-carbamoylamino acid, comprising contacting a hydantoin with the the rec-hydantoinase of claim 9.

20. A method of producing an N-carbamoylamino acid, comprising contacting a hydantoin with the the rec-hydantoinase of claim 10.

21. A method of making an amino acid, comprising:
producing an N-carbamoylamino acid according to the method of claim 19, and
contacting the N-carbamoylamino acid with a carbamoylase.

22. A method of making an amino acid, comprising:
producing an N-carbamoylamino acid according to the method of claim 20, and
contacting the N-carbamoylamino acid with a carbamoylase.

23. A cell transformed with the nucleic acid of claim 11.

24. A cell transformed with the nucleic acid of claim 13.
